(19) United States
(12) Patent Application Publication
(10) Pub. No.: US 2006/0096603 A1
(43) Pub. Date: May 11, 2006

(54) Method for complex phalloplasty with minimal incision

(76) Inventors: Hak Ryong Choi, Seoul (KR); Hoon Seog Jeon, Seoul (KR); Joon Yong Kim, Seongnam-si (KR); Ji Seok Leem, Bucheon-si (KR); Kyung Hun Kim, Dong-gu (KR)

Correspondence Address: FLESHNER & KIM, LLP, P.O. BOX 221200, CHANTILLY, VA, 20153, US

(21) Appl. No.: 10/982,809

(22) Filed: Nov. 8, 2004

(51) Int. Cl. A61B 17/00

(52) U.S. Cl. 128/898

(57) ABSTRACT

Disclose is a method for complex phalloplasty with minimal incision. In the method, a penile skin part directly behind the glans is minimally incised in a transverse direction with a depth enough to expose a Buck's fascia, a penile enhancement object made of a dermal strip is inserted between the subglans margin and the tunica albuginea through the minimally incised penile skin part. Then, the incised part is widened by a retractor, thereby enabling a prepubic area to be visible. Then, a part of a fundiform ligament or a suspensory ligament is partially incised, fixed, and then sealed. The minimal incision reduces the surgery time, reduces occurrence of postoperative necrosis and inflammation, thereby enabling the patients to rapidly recover from the surgery, and reduces postoperative complications, thereby enhancing the patients' satisfaction.

### BACKGROUND OF THE INVENTION

1. Field of the invention

The present invention relates to a method for complex phalloplasty with minimal incision, and more particularly to a method for complex phalloplasty, which enables simultaneous surgeries for glans augmentation, penile lengthening and girth enhancement through a minimal incision of a part of a dorsal area on the patient's penis. The method allows simultaneous surgery for dorsal neurectomy in the penis in order to heal premature ejaculation when necessary.

2. Description of the Prior Art

According to most conventional surgery methods for girth enhancement and penile lengthening, a patient's penis is locally anesthetized, either the patient's entire penis is widely circumcised or the patient's prepubic area is widely incised, either a synthetic implant made from soft or solution silicon or a natural implant made from cartilage, fat, dermofat or dermal tissue obtained from the patient himself or animals is inserted in a desired part, and the incised area is then sealed. Further, a silicon ring or silicon sheet, which is relatively inexpensive, is usually employed as the implant inserted in the penis. However, such conventional surgery methods using silicon insertion have not secured medical verification for safety of the silicon insertion yet and have caused more ill effects than the autologous dermal graft surgery. Nevertheless, the silicon insertion method is usually employed in the penile enlargement surgery, since the method is relatively easier, requires less cost, and satisfies the patient more.

Further, according to the conventional girth enhancement surgery using autologus dermofat graft, allograft dermal matrix, or xenograft, either the patient's entire penis is widely circumcised or the patient's prepubic area is widely incised, so that the surgery requires a long time. Further, the circumcision of a wide area of the patient's penile surface may leave scar on the area after the surgery and cause more complications, thereby causing complaints from the patient. Further, the incision of a wide area of the patient's prepubic area may cause the trouble of skin healing after the surgery.

### SUMMARY OF THE INVENTION

Accordingly, the present invention has been made to solve the above-mentioned problems occurring in the prior art, and an object of the present invention is to provide a method for complex phalloplasty with minimal incision, which can simultaneously achieve penile lengthening, girth enhancement, and glans augmentation through a minimal circumcision.

According to the present invention for accomplishing this object, a penile skin part directly behind the glans is transversely incised with a depth of 0.3 to 0.5 cm and a length of 2 to 3 cm, so that an upper part of Buck's fascia is exposed and is then bluntly dissected up to prepubic area by the fingers. Thereafter, the transversely incised part is widened as much as possible by a small retractor so as to enable a part under the prepubic area to be visible. Then, a part of a fundiform ligament and a suspensory ligament are exposed and are incised and fixed. As a result, the incised suspensory ligament causes the corpus cavernosum to protrude 2 to 4 cm forward, thereby lengthening the exposed part of the penis.

In a simultaneous surgery for penile lengthening and glans augmentation according to the present invention, using the minimally incised part a sufficient space between subglans margin and tunica albuginea is secured by means of finger and iris scissor dissection. Then an enhancement tissue is placed on the space while preventing the penile enhancement tissue from necrotizing. Thereafter, the incised part is precisely sealed while preventing enhancement tissue from leaking out. In the surgery for glans augmentation, either autologus dermofat obtained from the patient's donor part such as hips or other biocompatible materials are uesed. Herein, the enhanced tissue is formed into a dermal strip shaped like a rope and is inserted into the space between subglans margin and the tunica albuginea. Then, the incised part is sealed, thereby fixing the enhanced tissue.

According to another aspect of the present invention, there is provided a surgery method, in which a distal part of the penis is minimally incised, and the skin layer is then separated from the Buck's fascia up to the base part of the penis, thereby securing a sufficient space. Then, a penile enhancement tissue is spread as wide and flat as possible by a skin expander and is attached to the Buck's fascia layer. Further the subglans space could be obtained for the glans enhancement. Then, the incised part is sealed and fixed.

### BRIEF DESCRIPTION OF THE DRAWINGS

The above and other objects, features and advantages of the present invention will be more apparent from the following detailed description taken in conjunction with the accompanying drawings, in which:

FIG. 1A illustrates circumcision around an entire penis according to a conventional penile enlargement surgery method;

FIG. 1B illustrates insertion of autologus dermofat or enhancement material into the circumcised penis shown in FIG. 1A;

FIG. 2 illustrates a minimal incision of a penile skin part directly behind the glans in a transverse direction according to a method for complex phalloplasty with minimal incision according to the present invention;

FIG. 3 illustrates a partially incised ligament under the minimally incised penile skin part directly behind the glans according to a method for complex phalloplasty with minimal incision according to the present invention;

FIG. 4 illustrates a penis after undergoing a surgical operation according to a method for complex phalloplasty with minimal incision according to the present invention;

FIG. 5A illustrates a donor (autologus dermofat) part of an individual from which a penile enhancement object is obtained according to a method for complex phalloplasty with minimal incision according to the present invention; and

FIG. 5B illustrates the penile enhancement object attached according to a method for complex phalloplasty with minimal incision according to the present invention.

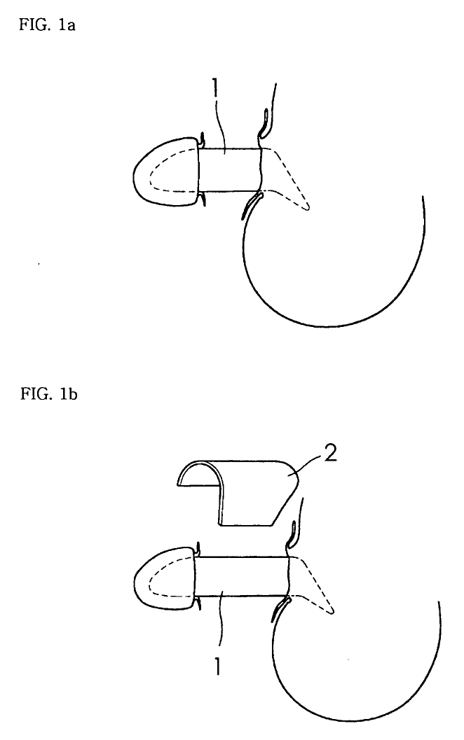

### DETAILED DESCRIPTION OF THE PREFERRED EMBODIMENTS

Hereinafter, preferred embodiments of the present invention will be described with reference to the accompanying drawings.

### EXAMPLE 1

Glans Augmentation

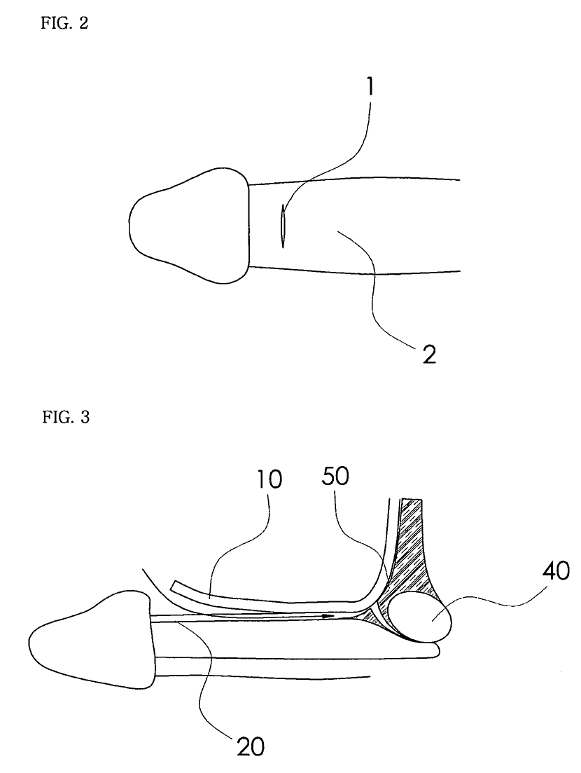

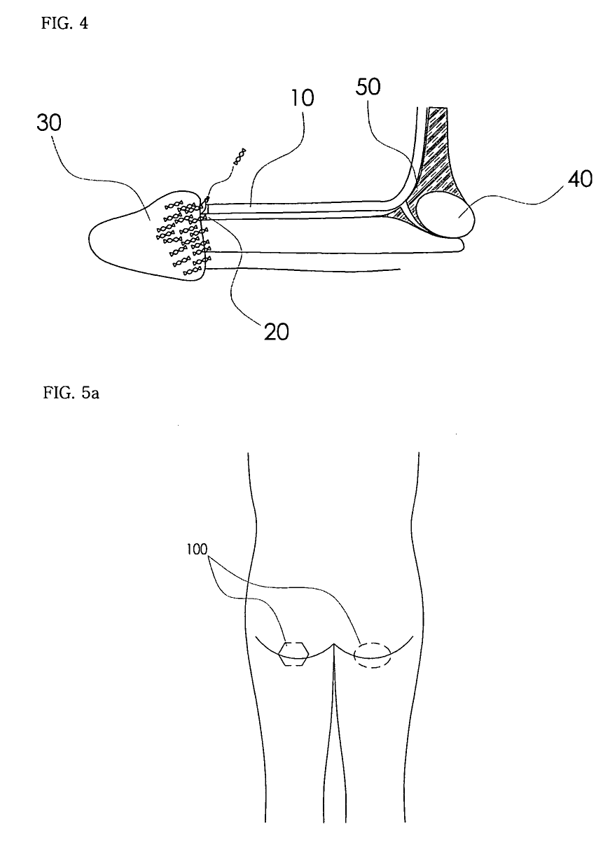

As shown in FIG. 2, a subdermal tissue of a patient's penile dorsal area 2 including the patient's cornal sulcus is locally anesthetized with 2% lidocaine. Thereafter, penile skin part 1 directly after the glans is transversely incised with a depth of 0.3 to 0.5 cm and a length of 2 to 3 cm, so that an upper part of Buck's fascia is exposed. Next, as shown in FIG. 4, the upper part directly above the Buck's fascia is incised so that the glans and the tunica albuginea are separated from each other. Then, penile enhancement tissues 30 made of dermal strips are interposed between the subglans margin and the tunica albuginea. Thereafter, surgical wound is sealed and the penis is then compressively dressed. Here, the enhancement tissues 30 have been usually made of autologus dermofat obtained from donor parts 100. Recently, the enhancement tissues 30 are often made from bio-compatible materials such as allograft dermal matrix (with a trademark “alloderm”) and collagen. However, more research regarding the efficiency and side effects of the biocompatible materials is necessary in order to employ the biocompatible materials as the penile enhancement tissues 30.

### EXAMPLE 2

Penile Lenghtening

Known methods for lengthening a penis include various skin manipulations, such as V-Y plasty, Z-plasty, double Z-plasty and M-plasty of a base part of a penis, and an adipectomy in the prepubis

In a surgery method according to the present invention in contrast to the known methods, the transversely incised part through which the Buck's fascia is exposed as in example 1 is widened as much as possible by a small retractor so as to enable a part under the pubis 40 to be visible. Then, a part of a fundiform ligament or a suspensory ligament 50 firmly coupled to the pubis is exposed. Thereafter, only a part of the suspensory ligament 50 is incised as shown in FIG. 3. Thereafter, in order to prevent re-bonding of the suspensory ligament 50 or penile shaft retraction, an insert such as an external tissue or gore-tex or autologous dermofat graft is inserted between the pubis 40 and the base part of the penis or the distal part of the incised suspensory ligament 50 is fixed to the dermal layer of the pubic area.

### EXAMPLE 3

Girth Enhancement

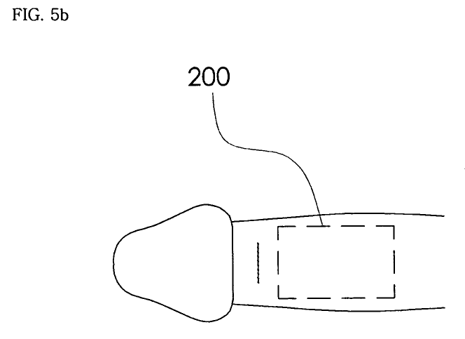

First, dermofat is prepared mainly by obtaining autologous dermofat from a donor part such as the patient's hips (as shown in FIG. 5), abdomen, waist, or groin after local anesthesia with 1%. Preferably, the obtained autologous dermofat has a shape of an ellipsoidal strip or elongated hexagonal strip 100 having a length of 7 to 10 cm and a width of 3 to 5 cm. After obtaining, Epidermis is eliminated from the strip and the autologous dermofat is stored within physiological saline. Here, the extracted autologous dermofat strip cannot have a thickness exceeding 1 cm. Thereafter, the tissue of the incised donor site 100 is carefully sealed.

Next, the area between the glans and the base part of the penis is locally anesthetized with 1:1 mixture solution of 1% lidocaine and bupivacaine, a distal part of the penis is minimally incised, and the skin and the subdermal layer are then separated from the Buck's fascia described in example 1 up to the base part of the penis. The obtained autologous dermofat, i.e. penile enhancement tissue, is fixed with 3-0 chrome catgut. Then, as shown in FIG. 5B, the penile enhancement tissue 30 is spread as wide and flat as possible by a skin expander (not shown) and is attached so that the fat layer is oriented toward the Buck's fascia layer. Then, the incised part is sealed and fixed while preventing the autologous dermofat from protruding outward. The dermal matrix allograft or other biocompatible material is carried out in the same way as described above.

Surgeries according to examples 1, 2, and 3 of the present invention as described above have showed the following clinical results.

The surgeries were performed for 660 patients having a small penis complex in hospitals located in Republic of Korea from January of 2003 to July of 2004. The patients were at ages of twenty to fifty eight, and mainly were thirties and forties. The patients's penises had lengths of 3 to 7 cm (average 5.2 cm) before the surgery and showed length increase of 1.5 to 5 cm (average 2.5 cm) through the surgery of example 2. Further, through surgeries of example 1 and 3, circumferences of the penises showed increases of 2.8 to 6.8 cm (average 4.8 cm) and diameters of glanses showed increases of 0.6 to 0.7 cm. Further, the surgeries of the present invention showed minimized isolation between the glans and trunk of the penis. Also, the surgeries of the present invention including surgery on the donor part of the autologous dermofat took only thirty minutes to one hour, which is one to three hours shorter than surgeries according to the conventional methods. Moreover, according to the surgeries of the present invention, the incised part was minimized, so that postoperative scars, skin necrosis, and wound infection were considerably reduced in comparison with the case of circumcising the entire penis.

The present invention is more advantageous than the conventional surgery methods in that:

First, the transverse incision of a minimal part of a penis directly behind the glans employed in the present invention reduces the surgery time;

Second, the minimized incised part reduces occurrence of postoperative complications, thereby enabling the patients to rapidly recover from the surgery, and reduces postoperative scars, thereby enhancing the patients' satisfaction;

Third, the surgery method of the present invention enables simultaneous surgeries for glans augmentation, penile lengthening and girth enhancement.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A method for complex phalloplasty with minimal incision, the method comprising the steps of:
(1) minimally incising a penile skin part directly behind the glans in a transverse direction with a depth enough to expose a Buck's fascia;
(2) inserting a penile enhancement object made of a dermal strip between the subglans margin and the tunica albuginea through the minimally incised penile skin part;
(3) widening the incised part by a retractor so as to enable a prepubic area to be visible; and
(4) partially incising, fixing, and then sealing a part of a fundiform ligament or a suspensory ligament.

2. The method as claimed in claim 1, further comprising the steps of:
after step (1), separating a skin and an epidermal layer from the Buck's fascia up to a base part of a penis, thereby securing a sufficient space;
spreading a penile enhancement tissue as wide and flat as possible by a skin expander;
attaching the penile enhancement tissue so that a fat layer of the penile enhancement tissue is oriented toward the Buck's fascia layer; and
sealing and fixing the incised part.

3. The method as claimed in claim 1, wherein the penile enhancement tissue is one of autologous dermofat, dermal matrix allograft, or other biocompatible material.

4. The method as claimed in claim 1, wherein the minimally behind the glans and has a length of 2 to 3 cm in the incised penile skin part is located 0.3 to 0.5 cm directly transverse direction.
